(19) United States
(12) Patent Application Publication
(10) Pub. No.: US 2014/0378838 A1
(43) Pub. Date: Dec. 25, 2014

(54) APPARATUS FOR BEAMFORMING INCLUDING MULTI-CLUSTER ARCHITECTURE

(71) Applicant: SAMSUNG ELECTRONICS CO., LTD, Suwon-si (KR)

(72) Inventors: Chang Yong SON, Gunpo-si (KR); Do Hyung KIM, Hwaseong-si (KR); Min Soo KIM, Yongin-si (KR); Kang Eun LEE, Hwaseong-si (KR); Shi Hwa LEE, Seoul (KR)

(73) Assignee: SAMSUNG ELECTRONICS CO., LTD., Suwon-si (KR)

(21) Appl. No.: 14/308,920

(22) Filed: Jun. 19, 2014

(30) Foreign Application Priority Data
Jun. 19, 2013 (KR) 10-2013-0070372

(51) Int. Cl. A61B 8/00

(52) U.S. Cl. CPC A61B 8/4281; A61B 8/54
USPC 600/447

(57) ABSTRACT

An apparatus for beamforming including a multi-cluster architecture is disclosed, in which, a number of unit clusters including a reconfigurable processor and a co-processor may vary based on requirements.

### CROSS-REFERENCE TO RELATED APPLICATIONS

This application claims the benefit under 35 U.S.C. §119(a) of Korean Patent Application No. 10-2013-0070372, filed on Jun. 19, 2013, in the Korean Intellectual Property Office, the entire disclosure of which is incorporated herein by reference for all purposes.

### BACKGROUND

1. Field

The following description relates to an apparatus and method for beamforming including an expandable multi-cluster architecture.

2. Description of Related Art

Existing ultrasonic diagnosis apparatuses may be suitable for real-time processing through use of hardware dedicated to obtaining an ultrasonic diagnosis. For example, the existing ultrasonic diagnosis apparatus may implement all functions through an application specific integrated circuit (ASIC) and a field programmable gate array (FPGA). However, high costs and a long time required to adjust to changes in a medical application have been associated with existing ultrasonic diagnosis apparatus due to difficulty in adapting a medium based on processing a fixed parameter to applying a newly developed algorithm to the existing ultrasonic diagnosis apparatus.

With development of processors, such as, for example, digital signal processor (DSP), a graphics processing unit (GPU), and a central processing unit (CPU), however, some functions of signal processing, image processing, and the like, aside from beamforming of the ultrasonic diagnosis apparatus, are being implemented using such processors. Using such new processors may not only reduce a period and costs associated with development, but also enable instant application of a new algorithm.

However, configuring a software-based system using an existing DSP, GPU, CPU, and the like, may be difficult for beamforming, which requires a large volume of data operation and a relatively broad data bandwidth in an ultrasonic diagnosis apparatus.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In one general aspect, there is provided an apparatus for beamforming, including at least one unit cluster configured to include a plurality of configurable processors for beamforming ultrasonic data.

A number of the at least one unit cluster may be configured to vary based on a number of beamforming channels or a frame ratio, or both the number of beamforming channels and the frame ratio.

A number of the at least one unit cluster may be configured to vary in response to the use of an adaptive algorithm for beamforming.

The at least one unit cluster may include a plurality of unit clusters, and the apparatus may further comprise a controller configured to load an identical program to the unit clusters to perform an identical operation in a scan line unit of ultrasonic data.

The apparatus may include a memory processor configured: to control ultrasonic data stored in a memory based on a scan line unit of ultrasonic data to be provided to the at least one unit cluster.

The at least one unit cluster may comprise a plurality of reconfigurable processors for processing ultrasonic data corresponding to one scan line, in a depth bundle unit.

The at least one unit cluster may further comprise a co-processor for performing a multiplication and accumulation (MAC) operation or a multiplication (MUL) operation in parallel on the ultrasonic data processed by the plurality of reconfigurable processors.

The apparatus may include a memory processor configured to divide the ultrasonic data stored in a memory and to provide the divided ultrasonic data to the at least one unit cluster, and to store a result of the beamforming performed by the at least one unit cluster in the memory.

A number of the at least one unit cluster may be configured to vary based on a number of beamforming channels or a frame ratio, or both the number of beamforming channels and the frame ratio.

A number of the at least one unit cluster may be configured to vary in response to the use of an adaptive algorithm for beamforming.

The apparatus may include a controller configured to schedule ultrasonic data based on a scan line; and at least one unit cluster configured to perform beamforming, using the scheduled ultrasonic data.

The at least one unit cluster may comprise a plurality of reconfigurable processors configured to process the scheduled ultrasonic data in a depth bundle unit.

The at least one unit cluster may comprises a co-processor that performs, in parallel, a multiplication and accumulation (MAC) operation or a multiplication (MUL) operation on ultrasonic data processed by the plurality of reconfigurable processors.

The at least one unit cluster may comprise a plurality of unit clusters, and the controller is configured to load an identical program to the unit clusters for an identical operation to be performed on the unit clusters.

In another general aspect, there is provided an apparatus for beamforming including a memory processor configured to divide the ultrasonic data stored in a memory and to provide the divided ultrasonic data to at least one unit cluster for beamforming; a controller configured to schedule the divided ultrasonic data for beamforming; and the at least one unit cluster is configured to include a plurality of configurable processors for beamforming the scheduled ultrasonic data.

A number of the at least one unit cluster may be configured to vary based on a requirement of an ultrasonic diagnosis apparatus.

The requirement of the ultrasonic diagnosis apparatus may include at least one of a number of beamforming channels, a frame ratio, and whether an adaptive algorithm requiring a high-complexity is used.

The at least one unit cluster may comprise a plurality of reconfigurable processors configured to process ultrasonic data, corresponding to one scan line, in a depth bundle unit; and a co-processor configured to perform, in parallel, a multiplication and accumulation (MAC) operation or a multiplication (MUL) operation on the ultrasonic data processed by the plurality of reconfigurable processors.

The co-processor may include an address generator configured to transfer data between the co-processor and a sub-system bus; a scratchpad memory (SPM) configured to store a result processed by the co-processor; a wide-way MAC configured to calculate a mass volume ultrasonic data, in parallel; and a combox configured to control the wide-way MAC.

The memory processor may be further configured to store a result of the beamforming performed by the at least one unit cluster in the memory.

Other features and aspects will be apparent from the following detailed description, the drawings, and the claims.

### BRIEF DESCRIPTION OF THE DRAWINGS

FIG. 1 is a diagram illustrating an example of an operation of an apparatus for beamforming.

FIG. 2 is a diagram illustrating an example of a detailed configuration of an apparatus for beamforming.

FIG. 3 is a diagram illustrating an example of a detailed configuration of a reconfigurable processor (RP) cluster of FIG. 2.

FIG. 4 is a diagram illustrating an example of ultrasonic data processed by an RP cluster.

FIG. 5 is a diagram illustrating an example of expanding an RP cluster.

FIG. 6 is a diagram illustrating an example of a co-processor included in an RP cluster.

FIG. 7 is a diagram illustrating an example of an operation of a co-processor included in an RP cluster.

FIG. 8 is a diagram illustrating an example of a process in which a co-processor performs a large volume of multiplication (MUL) operation.

Throughout the drawings and the detailed description, unless otherwise described or provided, the same drawing reference numerals will be understood to refer to the same elements, features, and structures. The drawings may not be to scale, and the relative size, proportions, and depiction of elements in the drawings may be exaggerated for clarity, illustration, and convenience.

### DETAILED DESCRIPTION

The following detailed description is provided to assist the reader in gaining a comprehensive understanding of the methods, apparatuses, and/or systems described herein. However, various changes, modifications, and equivalents of the systems, apparatuses, and/or methods described herein will be apparent to one of ordinary skill in the art. The progression of processing steps and/or operations described is an example; however, the sequence of and/or operations is not limited to that set forth herein and may be changed as is known in the art, with the exception of steps and/or operations necessarily occurring in a certain order. Also, descriptions of functions and constructions that are well known to one of ordinary skill in the art may be omitted for increased clarity and conciseness.

The features described herein may be embodied in different forms, and are not to be construed as being limited to the examples described herein. Rather, the examples described herein have been provided so that this disclosure will be thorough and complete, and will convey the full scope of the disclosure to one of ordinary skill in the art.

An apparatus for processing beamforming may refer to a programmable apparatus to be implemented in a flexibly reconfigurable and expandable cluster architecture, which is configured to focus ultrasonic data received from an ultrasonic transducer on a plurality of image points. A unit cluster for the expandable architecture for processing beamforming may include a plurality of reconfigurable processors including a co-processor and a multi-core processor for beamforming that process a high-efficiency algorithm in real time.

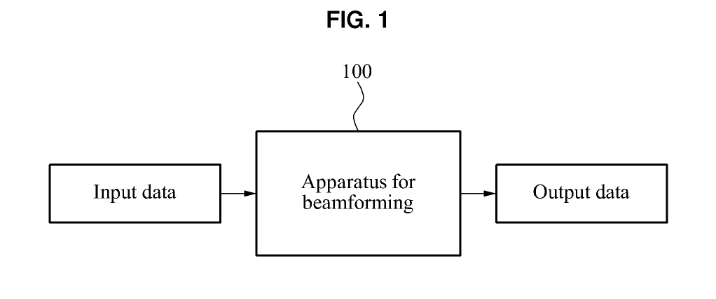

FIG. 1 illustrates an example of an operation of an apparatus 100 for beamforming. Referring to FIG. 1, the apparatus 100 for beamforming may receive input data, process input data, and produce output data. The apparatus 100 for beamforming may be an integral portion of an ultrasonic diagnosis apparatus, or may be configured separately from the ultrasonic diagnosis apparatus. The input data may indicate ultrasonic data received from an ultrasonic converter. The output data may indicate the ultrasonic data on which beamforming is performed.

Ultrasonic data for beamforming may require a large volume of data operations and a relatively broad data bandwidth. Accordingly, the apparatus 100 for beamforming may be flexibly such that it may be expanded to a multi-cluster architecture based on a requirement of the ultrasonic diagnosis apparatus. The requirement of the ultrasonic diagnosis apparatus may include a number of beamforming channels, a required frame ratio, and whether an adaptive algorithm requiring a high complexity is used.

The apparatus 100 for beamforming may store a scan line of ultrasonic data and a unit scan line in an internal memory for parallel processing based on a sampling depth. The apparatus 100 for beamforming may have a unit cluster architecture for minimizing overlapping requests for access based on depth. The apparatus 100 for beamforming may process multiple core processors through parallelization for an efficient distribution of the ultrasonic data.

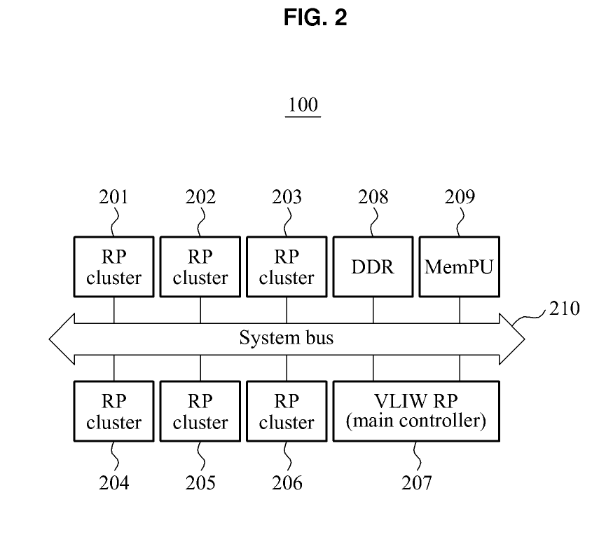

FIG. 2 illustrates an example of a detailed configuration of an apparatus 100 for beamforming. Referring to FIG. 2, the apparatus 100 for beamforming may include reconfigurable processor (RP) clusters 201, 202, 203, 204, 205, and 206, a memory 208, a memory processor (MemPU) 209, a very long instruction word reconfigurable processor (VLIW RP) 207, and a system bus 210.

The apparatus 100 for beamforming may have a multi-cluster architecture that may adaptively expand the RP clusters 201 through 206, based on requirements of the ultrasonic diagnosis apparatus. In the example illustrated in FIG. 2, six RP clusters 201 through 206 are shown. However, based on the requirements of the ultrasonic diagnosis apparatus, a number of RP clusters may be varied from at least one to N without departing from the spirit and scope of the illustrative examples described.

The RP clusters 201 through 206 refer to a unit cluster including reconfigurable processors, which may be a basic unit for performing beamforming. The RP clusters 201 through 206 may process ultrasonic data in parallel. The reconfigurable processors included in the RP clusters 201 through 206 may process ultrasonic data in a depth bundle unit, and a co-processor may process the ultrasonic data processed by the reconfigurable processors in parallel.

The MemPU 209 refers to a memory processor that may efficiently store ultrasonic data in the memory 208. The MemPU 209 may store a result of beamforming performed by the RP clusters 201 through 206 in the memory 208 through the system bus 210. The MemPU 209 may divide the ultrasonic data stored in the memory 208 and may provide the divided ultrasonic data to the RP clusters 201 through 206 through the system bus 210. The VLIW RP 207 refers to a controller for controlling an entire parallel processing program of the apparatus 100 for beamforming.

The apparatus 100 for beamforming may include a unit cluster configured by a co-processor and a multi-processor. Based on a desired function and the system requirements, a number of physically identical clusters may be increased.

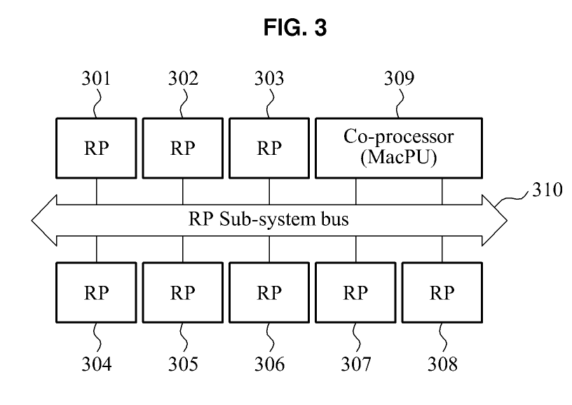

FIG. 3 illustrates an example of a detailed configuration of an RP cluster of FIG. 2. The RP cluster may include a plurality of RPs 301, 302, 303, 304, 305, 306, 307, and 308, a co-processor (MacPU) 309, and a sub-system bus 310. Based on the system requirements, a number of RPs may be varied from at least one to N without departing from the spirit and scope of the illustrative examples described. The RP cluster may be referred to as a basic unit for beamforming. The RPs 301 through 308 refers to a reconfigurable processor, and may be implemented by processor, such as, for example, a high efficiency digital signal processor (DSP). The RP cluster may indicate a multi-processor architecture configured by the plurality of RPs 301 through 308.

A result processed by the plurality of RPs 301 through 308 may be transferred to a co-processor 309 through the sub-system bus 310. The co-processor (MacPU) 309 may perform an arithmetic operation on the ultrasonic data processed by the plurality of RPs 301 through 308 in parallel. The co-processor (MacPU) 309 may perform a multiplication and accumulation (MAC) operation or a multiplication (MUL) in parallel on the ultrasonic data processed by the plurality of RPs 301 through 308.

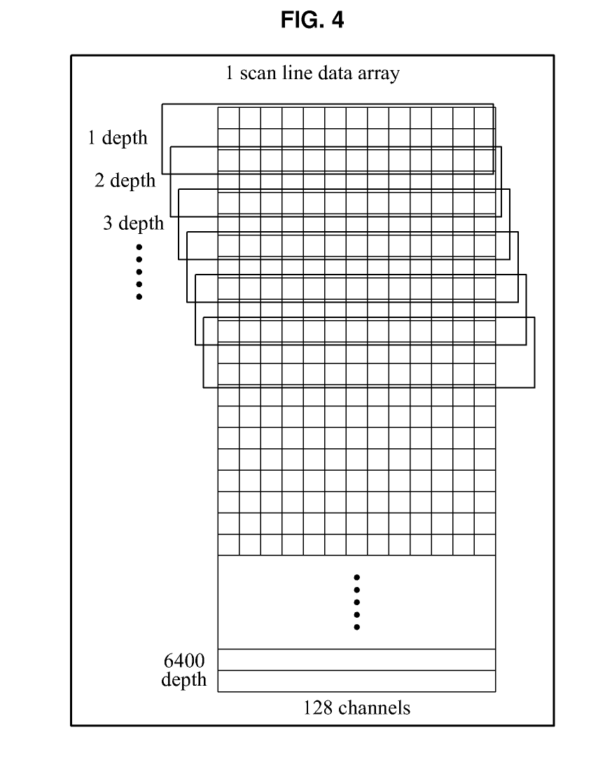

FIG. 4 illustrates an example of ultrasonic data processed by an RP cluster. Referring to FIG. 4, a VLIW RP included in the apparatus 100 for beamforming may schedule ultrasonic data for a plurality of scan lines, and transfer the ultrasonic data to a plurality of RP clusters. The plurality of RP clusters may store unit data for one scan line in a memory included in the plurality of RP clusters, and process the stored unit data.

RPs included in the plurality of RP clusters may process the unit data in one scan line in a depth bundle unit. The RPs included in the plurality of RP clusters may process a 1 depth bundle, a 2 depth bundle, and a 3 depth bundle configuring one scan line of data, as illustrated in FIG. 4. For example, the plurality of RPs included in the plurality of RP clusters may process ultrasonic data in a depth bundle unit, for example, a k-depth/p-processor.

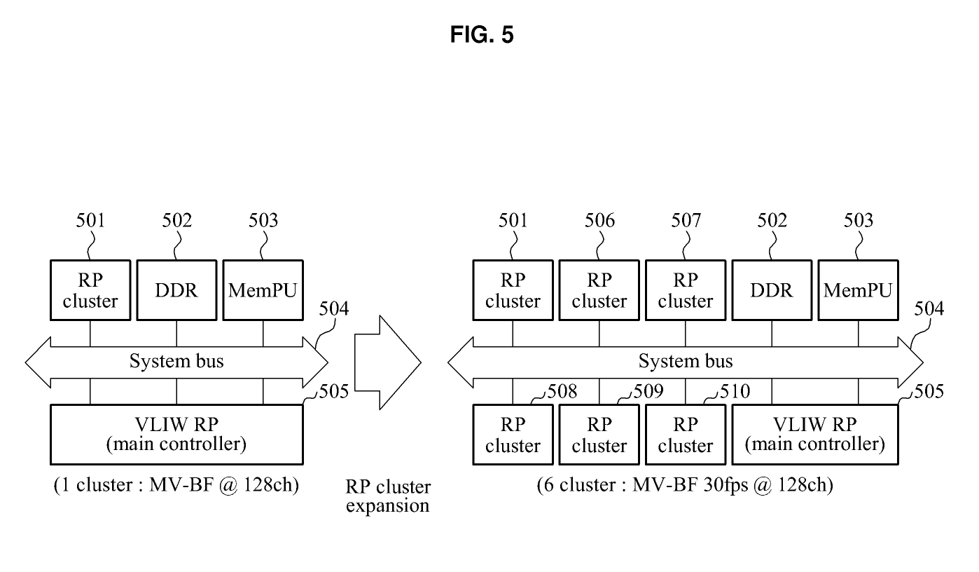

FIG. 5 illustrates an example of expanding an RP cluster. Referring to FIG. 5, the apparatus 100 for beamforming may include a single RP cluster 501. Referring to FIG. 5, the apparatus 100 for beamforming may also include a memory 502, a memory processor (MemPU) 503, a very long instruction word reconfigurable processor (VLIW RP) 505, and a system bus 504. The system bus 504 may be implemented using architecture and configurations, such as, for example, Advanced Microcontroller Bus Architecture (AMBA) system-on-a-chip (SoC) design, AMBA 3 including AXI, AMBA 4 AXI4, AMBA 4 ACE, and AMBA 5 CHI (Coherent Hub Interface). Here, the apparatus 100 for beamforming is assumed to process ultrasonic data for minimum variance beamforming (MV-BF) of 128 channels. When the ultrasonic data of 128 channels is to be processed in a frame ratio of 30 frames per second (fps), the apparatus 100 for beamforming may increase a number of RP clusters. The apparatus 100 for beamforming may process 1 scan line data, using six RP clusters 501, 506, 507, 508, 509, and 510.

The apparatus 100 for beamforming may scalably configure a number of RP clusters, based on requirements of the ultrasonic diagnosis apparatus. The requirements the ultrasonic diagnosis apparatus may include, but is not limited to, a number of beamforming channels, a required frame ratio, and whether an adaptive algorithm requiring a high complexity is used.

For one example, in the apparatus 100 for beamforming, the VLIW RP 505 may load an identical program to the RP clusters 501, 506, 507, 508, 509, and 510, such that an identical arithmetic operation may be performed on the RP clusters 501, 506, 507, 508, 509, and 510. In beamforming the ultrasonic data, the RP clusters for example, unit clusters 501, 506, 507, 508, 509, and 510, may need to equally share a program code because an identical operation is performed in a scan line unit.

Accordingly, the VLIW RP 505 may control a program code to be automatically expanded based on a number of the RP clusters 501, 506, 507, 508, 509, and 510, and control the program code to be identically processed on the RP clusters 501, 506, 507, 508, 509, and 510.

The VLIW RP 505 may sequentially provide the ultrasonic data in a scan line to the RP clusters 501, 506, 507, 508, 509, and 510, based on the number of RP clusters 501, 506, 507, 508, 509, and 510. To this end, the VLIW RP 505 may control the MemPU 503, and provide the ultrasonic data stored in the memory 502 to the RP clusters 501, 506, 507, 508, 509, and 510. The RP clusters 501, 506, 507, 508, 509, and 510 may be controlled by a combox present in an interior of the VLIW RP 505.

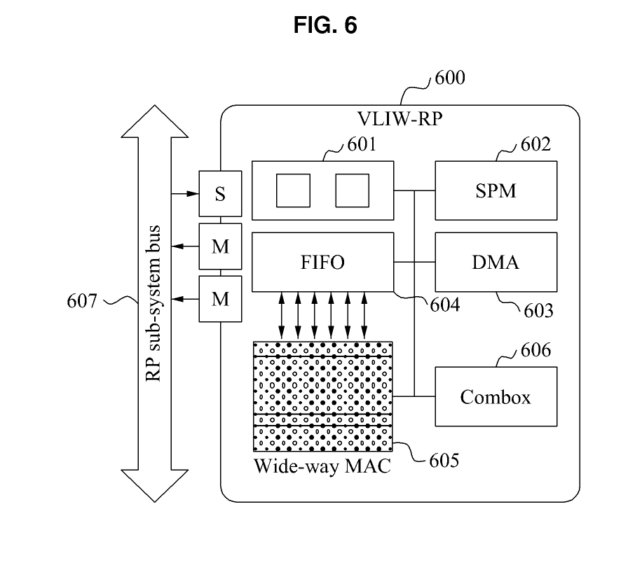

FIG. 6 illustrates an example of a co-processor (or the MacPU) included in an RP cluster. When the apparatus 100 for beamforming performs beamforming on ultrasonic data, an arithmetic operation required most frequently may be a large volume of multiplication or an MAC operation, such as convolution, filtering, correlation, weighted sum, or matrix operation. The apparatus 100 for beamforming may use a co-processor for rapidly processing a large volume of ultrasonic data in parallel. The co-processor may be included in an RP cluster of the apparatus 100 for beamforming.

Referring to FIG. 6, a VLIW-RP co-processor 600 may include an address generation unit 601, a scratchpad memory (SPM) 602, a direct memory access (DMA) 603, a first-in first-out FIFO buffer 604, a wide-way MAC unit 605, and a combox 606.

The co-processor 600 may control performing of a programmable operation. The address generation unit 601 may transfer data between the co-processor 600 and the sub-system bus 607. The SPM 602 may store a result processed by the co-processor 600. The DMA 603 may control transmission/reception of data through the MAC unit 605 connecting to the FIFO buffer 604. The MAC unit 605 may calculate the mass volume ultrasonic data in parallel.

The combox 606 may support an operation code (opcode) for programmably controlling the MAC unit 605. The address generation unit 601 may provide an input/output function of flexible data for the MAC unit 605 to rapidly process parallel calculation of mass volume ultrasonic data. The co-processor 600 may perform an MAC operation or MUL operation in parallel on the mass volume ultrasonic data, based on software.

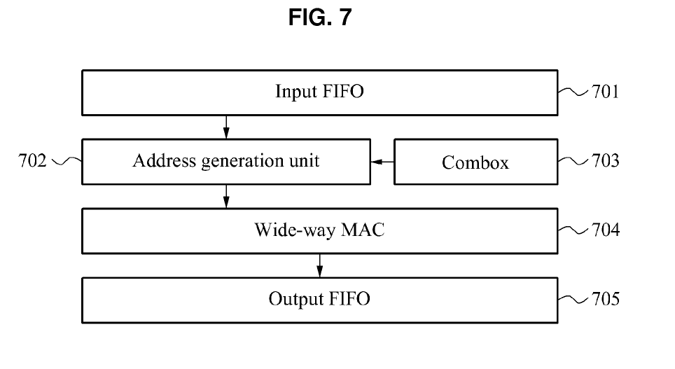

FIG. 7 illustrates an example of an operation of a co-processor included in an RP cluster. FIG. 7 illustrates an operation of an address generation unit 702 in a co-processor. The address generation unit 702 may transfer input data to a wide-way MAC unit 704 for performing an MAC operation in parallel on a large volume of ultrasonic data. Also, the address generation unit 702 may automatically provide data to the wide-way MAC unit 704 from an input FIFO 701, and store a result of processing performed by the wide-way MAC unit 704 in an output FIFO 705, based on information of the combox 703 for a programmable operation. The address generation unit 702 may inform the combox 703 of progress in a processing state of the MAC unit 704.

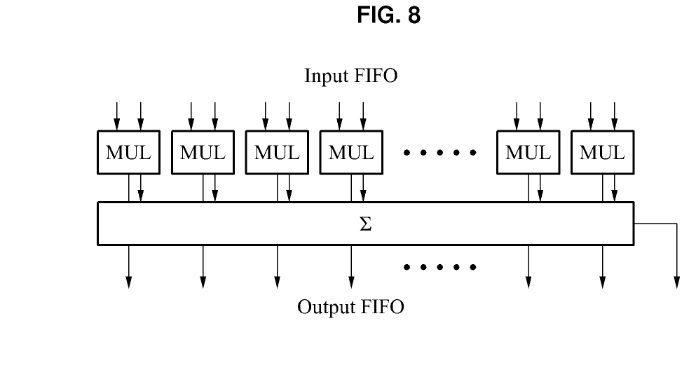

FIG. 8 illustrates an example of a process in which a co-processor performs a mass volume of multiplication (MUL) operation.

Referring to FIG. 8, an MAC unit included in the co-processor may perform an MAC operation or MUL operation on ultrasonic data processed by a plurality of RPs included in an RP cluster. The MAC unit may perform MAC and MUL operations in parallel, and rapidly process a large volume of ultrasonic data.

An apparatus for beamforming may process a high efficiency beamforming algorithm in real time based on software. The apparatus for beamforming may include a unit cluster including a co-processor for performing mass volume parallel MAC/MUL operation.

Also, the apparatus for beamforming may include a unit cluster configured by a plurality of RPs, such that parallel processing may be possible without dependency on a plurality of channels or scan lines of ultrasonic data in beamforming. A number of unit clusters may flexibly change based on a requirement of an ultrasonic diagnosis apparatus. The requirement of the ultrasonic diagnosis apparatus may include, for example, a number of beamforming channels, a frame ratio, and whether an adaptive algorithm requiring a high-complexity is used. The apparatus for beamforming may be expanded to a multi-cluster architecture.

The apparatus for beamforming may include a controller for distributing data into a plurality of unit clusters and for controlling the data. The apparatus for beamforming may also include a memory and a MemPU for storing and transferring mass volume ultrasonic data processed by a unit cluster.

Also, the co-processor included in the unit cluster refers to a processor having a 2 or 3-issue VLIW structure available for programming. The co-processor may communicate via a combox configured by a register, and include a hardware calculation unit, for example, a wide-way MAC, such that varied processing may be possible based on combox information. When the co-processor is exclusively used for mass volume calculation, it may reduce a cost of a remaining multi-processor and may enable real-time processing through parallel operation processing.

The apparatus for beamforming may include a unit cluster configured by a co-processor for supporting wide-way single instruction multiple data (SIMD), for example, a large volume of parallel operation processes, and a multi-core processor structure in order to ensure real-time with respect to a large volume of operation processing required by various high definition beamforming algorithms. Such a unit cluster may be scalable based on requirements, and thus be expanded to a multi-cluster architecture.

Accordingly, a number of unit clusters configured by the co-processor and the multi-processor may simply be increased, and a system satisfying requirements of a desired system may be reconfigured. Also, the unit cluster configured by a co-processor having a large volume parallel MAC hardware unit available for programming a beamforming function, and a multi-processor may reduce costs. Further, a beamforming performance may be enhanced through applying numerous software algorithms preciously inapplicable due to restrictions with respect to a hardware structure.

The processes, functions, and methods described above including a method for beamforming can be written as a computer program, a piece of code, an instruction, or some combination thereof, for independently or collectively instructing or configuring the processing device to operate as desired. Software and data may be embodied permanently or temporarily in any type of machine, component, physical or virtual equipment, computer storage medium or device that is capable of providing instructions or data to or being interpreted by the processing device. The software also may be distributed over network coupled computer systems so that the software is stored and executed in a distributed fashion. In particular, the software and data may be stored by one or more non-transitory computer readable recording mediums. The non-transitory computer readable recording medium may include any data storage device that can store data that can be thereafter read by a computer system or processing device. Examples of the non-transitory computer readable recording medium include read-only memory (ROM), random-access memory (RAM), Compact Disc Read-only Memory (CD-ROMs), magnetic tapes, USBs, floppy disks, hard disks, optical recording media (e.g., CD-ROMs, or DVDs), and PC interfaces (e.g., PCI, PCI-express, WiFi, etc.). In addition, functional programs, codes, and code segments for accomplishing the example disclosed herein can be construed by programmers skilled in the art based on the flow diagrams and block diagrams of the figures and their corresponding descriptions as provided herein.

The apparatuses and units described herein may be implemented using hardware components. The hardware components may include, for example, controllers, sensors, processors, generators, drivers, and other equivalent electronic components. The hardware components may be implemented using one or more general-purpose or special purpose computers, such as, for example, a processor, a controller and an arithmetic logic unit, a digital signal processor, a microcomputer, a field programmable array, a programmable logic unit, a microprocessor or any other device capable of responding to and executing instructions in a defined manner. The hardware components may run an operating system (OS) and one or more software applications that run on the OS. The hardware components also may access, store, manipulate, process, and create data in response to execution of the software. For purpose of simplicity, the description of a processing device is used as singular; however, one skilled in the art will appreciated that a processing device may include multiple processing elements and multiple types of processing elements. For example, a hardware component may include multiple processors or a processor and a controller. In addition, different processing configurations are possible, such a parallel processors.

While this disclosure includes specific examples, it will be apparent to one of ordinary skill in the art that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner and/or replaced or supplemented by other components or their equivalents. Therefore, the scope of the disclosure is defined not by the detailed description, but by the claims and their equivalents, and all variations within the scope of the claims and their equivalents are to be construed as being included in the disclosure.

What is claimed is:

1. An apparatus for beamforming, comprising:
at least one unit cluster configured to include a plurality of configurable processors for beamforming ultrasonic data.

2. The apparatus of claim 1, wherein a number of the at least one unit cluster is configured to vary based on a number of beamforming channels or a frame ratio, or both the number of beamforming channels and the frame ratio.

3. The apparatus of claim 1, wherein a number of the at least one unit cluster is configured to vary in response to the use of an adaptive algorithm for beamforming.

4. The apparatus of claim 1, wherein the at least one unit cluster comprises a plurality of unit clusters, and the apparatus further comprises a controller configured to load an identical program to the unit clusters to perform an identical operation in a scan line unit of ultrasonic data.

5. The apparatus of claim 1, further comprising a memory processor configured: to control ultrasonic data stored in a memory based on a scan line unit of ultrasonic data to be provided to the at least one unit cluster.

6. The apparatus of claim 1, wherein the at least one unit cluster comprise a plurality of reconfigurable processors for processing ultrasonic data corresponding to one scan line, in a depth bundle unit.

7. The apparatus of claim 6, wherein the at least one unit cluster further comprises a co-processor for performing a multiplication and accumulation (MAC) operation or a multiplication (MUL) operation in parallel on the ultrasonic data processed by the plurality of reconfigurable processors.

8. The apparatus of claim 1, further comprising a memory processor configured:
to divide the ultrasonic data stored in a memory and to provide the divided ultrasonic data to the at least one unit cluster; and
to store a result of the beamforming performed by the at least one unit cluster in the memory.

9. The apparatus of claim 1, wherein a number of the at least one unit cluster is configured to vary based on a number of beamforming channels or a frame ratio, or both the number of beamforming channels and the frame ratio.

10. The apparatus of claim 1, wherein a number of the at least one unit cluster is configured to vary in response to the use of an adaptive algorithm for beamforming.

11. An apparatus for beamforming, comprising:
a controller configured to schedule ultrasonic data based on a scan line; and
at least one unit cluster configured to perform beamforming, using the scheduled ultrasonic data.

12. The apparatus of claim 11, wherein the at least one unit cluster comprises a plurality of reconfigurable processors configured to process the scheduled ultrasonic data in a depth bundle unit.

13. The apparatus of claim 12, wherein the at least one unit cluster comprises a co-processor that performs, in parallel, a multiplication and accumulation (MAC) operation or a multiplication (MUL) operation on ultrasonic data processed by the plurality of reconfigurable processors.

14. The apparatus of claim 12, wherein the at least one unit cluster comprises a plurality of unit clusters, and the controller is configured to load an identical program to the unit clusters for an identical operation to be performed on the unit clusters.

15. An apparatus for beamforming, comprising:
a memory processor configured to divide the ultrasonic data stored in a memory and to provide the divided ultrasonic data to at least one unit cluster for beamforming;
a controller configured to schedule the divided ultrasonic data for beamforming; and
the at least one unit cluster is configured to include a plurality of configurable processors for beamforming the scheduled ultrasonic data.

16. The apparatus of claim 15, wherein a number of the at least one unit cluster is configured to vary based on a requirement of an ultrasonic diagnosis apparatus.

17. The apparatus of claim 16, wherein the requirement of the ultrasonic diagnosis apparatus comprises at least one of a number of beamforming channels, a frame ratio, and whether an adaptive algorithm requiring a high-complexity is used.

18. The apparatus of claim 15, wherein the at least one unit cluster comprises:
a plurality of reconfigurable processors configured to process ultrasonic data, corresponding to one scan line, in a depth bundle unit; and
a co-processor configured to perform, in parallel, a multiplication and accumulation (MAC) operation or a multiplication (MUL) operation on the ultrasonic data processed by the plurality of reconfigurable processors.

19. The apparatus of claim 18, wherein the co-processor comprises:
an address generator configured to transfer data between the co-processor and a sub-system bus;
a scratchpad memory (SPM) configured to store a result processed by the co-processor;
a wide-way MAC configured to calculate a mass volume ultrasonic data, in parallel; and
a combox configured to control the wide-way MAC.

20. The apparatus of claim 15, wherein the memory processor is further configured to store a result of the beamforming performed by the at least one unit cluster in the memory.
